(19) United States
(12) Patent Application Publication
(10) Pub. No.: US 2006/0257899 A1
(43) Pub. Date: Nov. 16, 2006

(54) Methods and Apparatus for Controlling DNA Amplification

(76) Inventors: William D. Bickmore JR., St. George, UT (US); Danvern Ray Roberts, Las Vegas, NV (US)

Correspondence Address: HOLME ROBERTS & OWEN, LLP, 299 SOUTH MAIN, SUITE 1800, SALT LAKE CITY, UT, 84111, US

(21) Appl. No.: 11/383,534

(22) Filed: May 16, 2006

Related U.S. Application Data
(62) Division of application No. 11/063,352, filed on Feb. 23, 2005.

(51) Int. Cl. C12Q 1/68; C12P 19/34; C12M 1/34

(52) U.S. Cl. 435/6; 435/91.2; 435/287.2

(57) ABSTRACT

Apparatus for optimizing DNA amplification are provided. A DNA cycler in accordance with the invention preferably includes a detector capable of measuring a parameter of at least one stage of DNA amplification that changes as the reaction of said stage approaches completion. It is presently preferred that fluorescence be used as such parameter. A microprocessor may be used to make comparisons of the value of the parameter over time, particularly as the reaction nears completion. A controller is used to terminate the current stage and progress to the next stage of DNA amplification when the reaction of the current stage has reached a desired state of completion. A method of the invention may include monitoring the progress of at least one stage of DNA amplification, measuring a parameter that provides data regarding the reaction of that stage, determining when the reaction of the stage has reached a desired level of completion, and the terminating the operation of the present stage and progressing to the next stage of the DNA amplification.

### CROSS-REFERENCE TO RELATED APPLICATIONS

This is a divisional application of application Ser. No. 11/063,352, filed on Feb. 23, 2005, entitled “Methods and Apparatus for Controlling DNA Amplification,” which is incorporated herein in its entirety and to which priority is claimed.

### BACKGROUND OF THE INVENTION

1. The Field of the Invention

The present invention is directed to a methods and apparatus for amplification of DNA.

2. The Relevant Technology

Molecular biology and associated applications rely heavily on the ability to manufacture large amounts of genetic material from small samples so that one can engage in activities such as the identification of particular genetic material in a sample, the measurement of how much genetic material was present, and generation of enough genetic material for use to serve as a component of further applications.

The most successful tool for this purpose is generally known as the “polymerase chain reaction” (PCR). The PCR process is generally performed in a small reaction vial containing components for DNA duplication: the DNA to be duplicated, the four nucleotides which are assembled to form DNA, two different types of synthetic DNA called “primers” (one for each of the complementary strands of DNA), salts, and an enzyme called DNA polymerase.

DNA is double stranded. The PCR process begins by separating the two strands of DNA into individual complementary strands, a step which is generally referred to as “denaturation.” This is typically accomplished by heating the PCR reaction mixture to a temperature of about 94 to about 96 degrees centigrade for a period of time between a few seconds to over a minute in duration.

Once the DNA is separated into single strands, the mixture is cooled to about 45 to about 60 degrees centigrade (typically chosen to be about 5 degrees below the temperature at which the primer will melt) in order to allow a primer to bind to each of the corresponding single strands of DNA in the mixture (this involves providing both “upstream” and “downstream” primers). This step is typically called “annealing.” The annealing step typically takes anywhere from a few seconds up to a few minutes.

Next, the reaction vessel is heated to about 72 to 73 degrees centigrade, a temperature at which DNA polymerase in the reaction mixture acts to build a second strand of DNA onto the single strand by adding nucleic acids onto the primer so as to form a double stranded DNA that is identical to that of the original strand of DNA. This step is generally called “extension.” The extension step generally takes from a few seconds to a couple minutes to complete.

This series of three steps, also sometimes referred to as “stages,” define one “cycle.” Completion of a PCR cycle results in doubling the amount of DNA in the reaction vial. Repeating a cycle results in another doubling of the amount of DNA in the reaction vial. Typically, the process is repeated many times, e.g. 10 to 40 times, resulting in a large number of identical pieces of DNA. Performing 20 cycles results in more than a million copies of the original DNA sample. Performing 30 cycles results in more than a billion copies of the original DNA sample. A “thermocycler” is used to automate the process of moving the reaction vessel between the desired temperatures for the desired period of time.

Conventional thermocyclers typically require about three hours to run 30 cycles, due to the amount of time accomplishing a change of temperature between each PCR step, as well as the time required at each target temperature. It would be of great interest in many situations if one could obtain the benefits of PCR more quickly than this.

More recently, thermocyclers have been made available that omit a separate extension stage, and operate as a two stage thermocycle. The first stage of this two stage system is denaturation, and the second stage is annealing, with extension occurring simultaneously with annealing.

### BRIEF SUMMARY OF THE INVENTION

The present invention provides methods and apparatus for amplification of DNA. More specifically, it has been discovered that the conventional practice of setting fixed times for each stage of a thermocycle does not provide optimal results in the amplification of the target DNA. A feature of the present invention is to monitor the state of the reaction of one or more stages of DNA amplification and terminating the present stage when the reaction reaches a desired state of completion.

The apparatus of the invention may include a DNA amplification cycler and a detector capable of measuring a parameter of at least one stage of DNA amplification that changes as the reaction of said stage approaches completion. It is presently preferred that fluorescence be used as such parameter. A microprocessor may be used to make comparisons of the value of the parameter over time, particularly as the reaction nears completion. A controller is used to terminate the current stage and progress to the next stage of DNA amplification when the reaction of the current stage has reached a desired state of completion.

A method of the invention may include monitoring the progress of at least one stage of DNA amplification, measuring a parameter that provides data regarding the reaction of that stage, determining when the reaction of the stage has reached a desired level of completion, and the terminating the operation of the present stage and progressing to the next stage of the DNA amplification.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a graphical representation of one cycle of a two stage DNA amplification process.

FIG. 2 is a graphical representation of fluorescent illumination over time in a DNA amplification experiment.

FIG. 3 is a graphical representation of one cycle of a two stage DNA amplification process.

FIG. 4 is a graphical representation of fluorescent illumination over time in a DNA amplification experiment that has not been optimized.

FIG. 5 is a graphical representation of fluorescent illumination over time in a DNA amplification experiment that has been optimized.

FIG. 6 depicts schematically one embodiment of the apparatus of the invention.

FIG. 7 is a flow chart of a microprocessor used to monitor a reaction in accordance with the present invention.

FIG. 8 is a flow chart for automatically determining the cycle count.

FIG. 9 is a graph of measured optimum times for each cycle of one DNA amplification experiment.

### DETAILED DESCRIPTION OF THE PREFERRED EMBODIMENTS

The present invention provides improved methods and apparatus for the amplification of DNA.

Conventional thermocyclers typically allow adjustments to be made only at the commencement of operation regarding the temperatures of each thermocycle stage, and the amount of time for each stage, and the total number of cycles to be run. All of these settings remain constant throughout the DNA amplification run.

The present invention has led to the discovery that this is not an optimum approach to effecting DNA amplification. More specifically, it has now been discovered that conventional thermocyclers typically cut off stages of a thermocycle prior to completion of the intended reaction, or allow the stage to run longer than required to complete the intended reaction. The present invention has also led to the discovery that the amount of time required to complete various stages of a thermocycler will change from cycle to cycle. This means that even if the time for a given stage is optimum for one cycle, it will not be optimum for other cycles. The present invention solves these problems of conventional thermocyclers.

It is possible to obtain the benefits of the present invention using either a three stage thermocycle or a two stage thermocycle, or even some alternative DNA amplification process. For purposes of brevity, the invention will be described by reference to a two stage thermocycler.

It is a feature of the present invention to monitor one or more of the stages in the DNA amplification process to determine when the reaction of a particular stage is complete, or as complete as desired, and then commence the next stage of DNA amplification. Although some benefit from this invention may be obtained by monitoring just one stage from cycle to cycle, greater benefit will typically be obtained by monitoring more than one step. The following discussion discusses application of the present invention with respect to both stages of a two stage thermocycler.

Various approaches may be used to monitor the steps of DNA amplification, but it is presently preferred to use fluorescence, and for purposes of brevity the present invention will be described by reference to use of fluorescence to monitor the amplification process. When using fluorescence to monitor DNA amplification, it is currently preferred to use the system of co-pending application Ser. No. 11/031,526 entitled “Fluorescence Detection System” and filed on Jan. 7, 2005, which is commonly assigned to the assignee of the present application, and which is incorporated herein by reference.

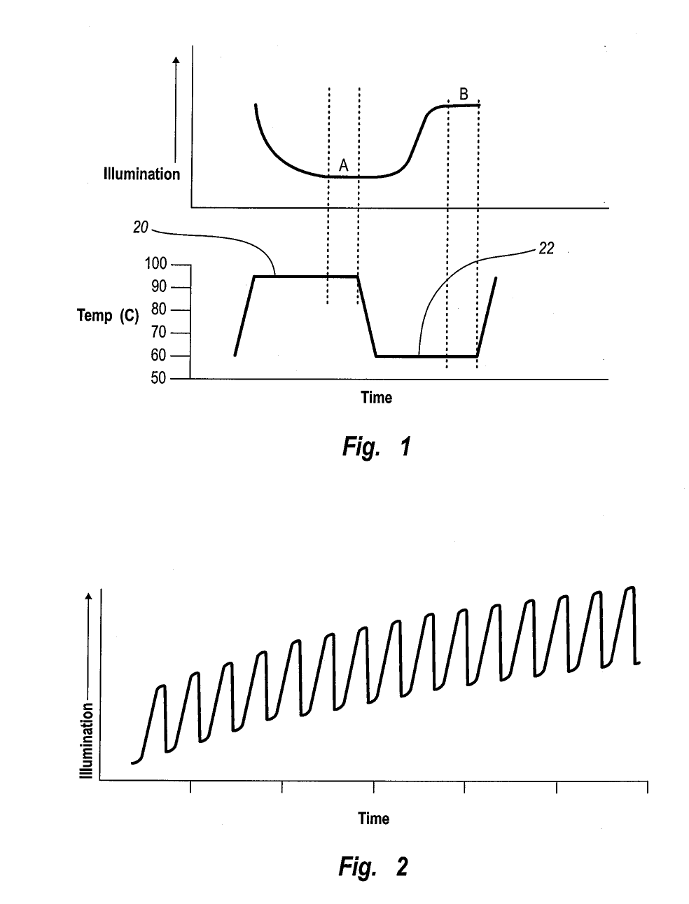

FIG. 1 is a two-part graph depicting one cycle of a two stage thermal cycle. The top graph of FIG. 1 shows fluorescent illumination over time, and the bottom graph of FIG. 1 shows the applied temperature over time. At the commencement of the cycle depicted in FIG. 1, the DNA is double stranded. By reference to the bottom graph, showing temperature over time, a denaturation stage 20 is depicted as occurring by raising the temperature to about 94 degrees centigrade. Double stranded DNA is separated into single stranded DNA during the course of the denaturation stage. It may be seen in the upper graph of FIG. 1 that fluorescent illumination decreases as double stranded DNA is denatured. If the temperature is held at 94 degrees long enough, denaturation will continue until all of the double stranded DNA is separated into single strands. Region A of FIG. 1 depicts a time when denaturation has been completed; although the temperature continues to be maintained at the denaturation temperature, the slope of fluorescent illumination in Region A has become substantially unchanging over time.

The opposite illumination effect is observed during annealing stage 22. After the temperature is reduced to about 60 degrees, the polymerase in the reaction mixture will construct double stranded DNA from the single strands of DNA in the reaction vial. As double stranded DNA is formed, the fluorescent illumination increases. If the annealing step is permitted to proceed long enough, all of the single stranded DNA will eventually be formed into double stranded DNA. Region B of FIG. 1 illustrates a time when the annealing process is complete, as shown by the substantially flat slope of the fluorescent illumination in that region.

It has been observed that subsequent thermocycles will result in ever increasing levels of fluorescent illumination when DNA amplification occurs. See FIG. 2. It has been observed that in cases where no DNA amplification occurs, the level of fluorescent illumination will be ever decreasing, which may be due to fluorescent decay due to the exposure of the fluorescent probe to a continuous light source.

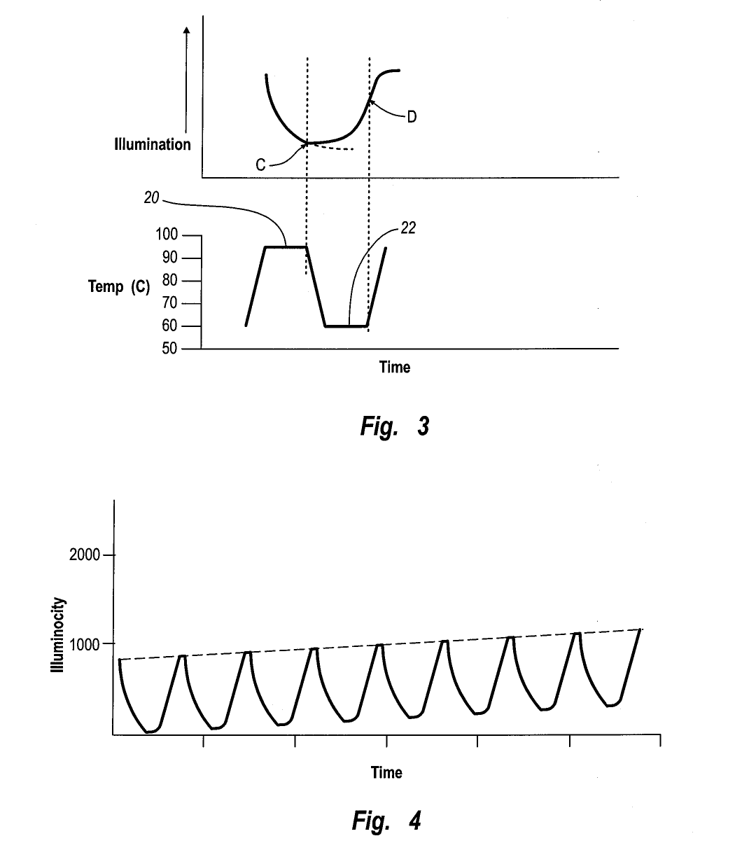

FIG. 3 depicts a situation where the stages are not permitted to run long enough to complete the desired reaction. FIG. 3 illustrates shortening of the denaturation and annealing stages at points C and D respectively. Point C illustrates the lowering of the temperature of the reaction mixture prior to denaturation of all of the DNA to single strands. Point D illustrates the increase of the temperature of the reaction mixture prior to all of the single strands of DNA being annealed and extended so as to form double stranded DNA. The effect is a sub-optimization of DNA amplification.

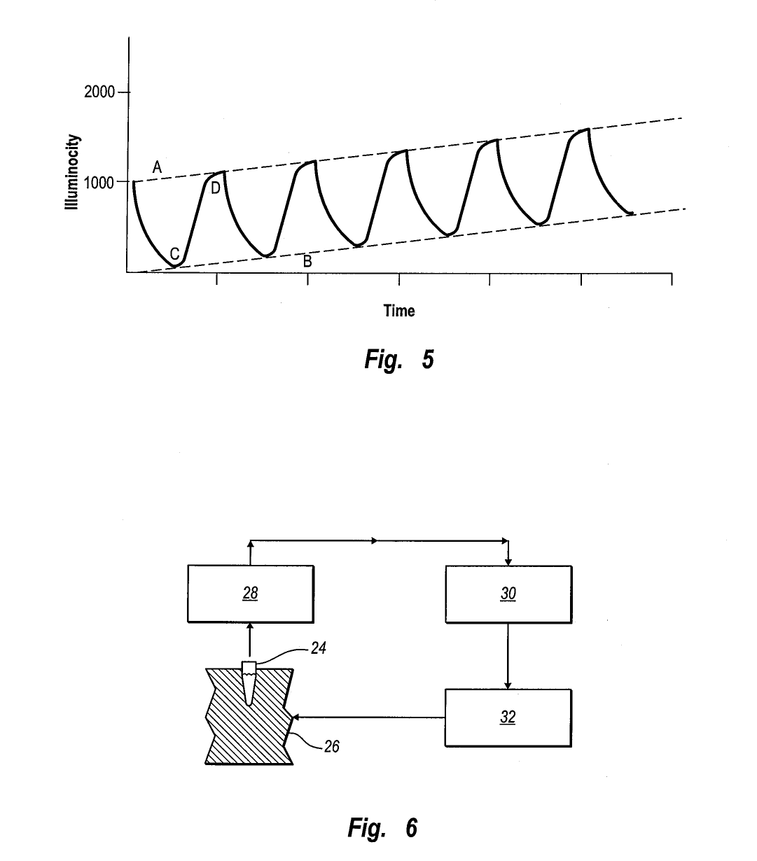

FIGS. 4 and 5 show the lesser efficiency resulting from failing to complete the reaction of the stages by comparison to completing the reaction at each stage. FIG. 4 depicts the situation where the overall cycle time is shorter than optimum, and FIG. 5 shows the situation where the overall cycle time has been optimized. The fluorescent illumination of FIG. 5 rises more rapidly over time than that of FIG. 4, illustrating that optimization in accordance with the present invention results in amplification of more DNA over time than if the cycle times are too short. Cycle times that are too long will also result in slower amplification, since no reactions are progressing during any interval when the reaction of a stage is already complete.

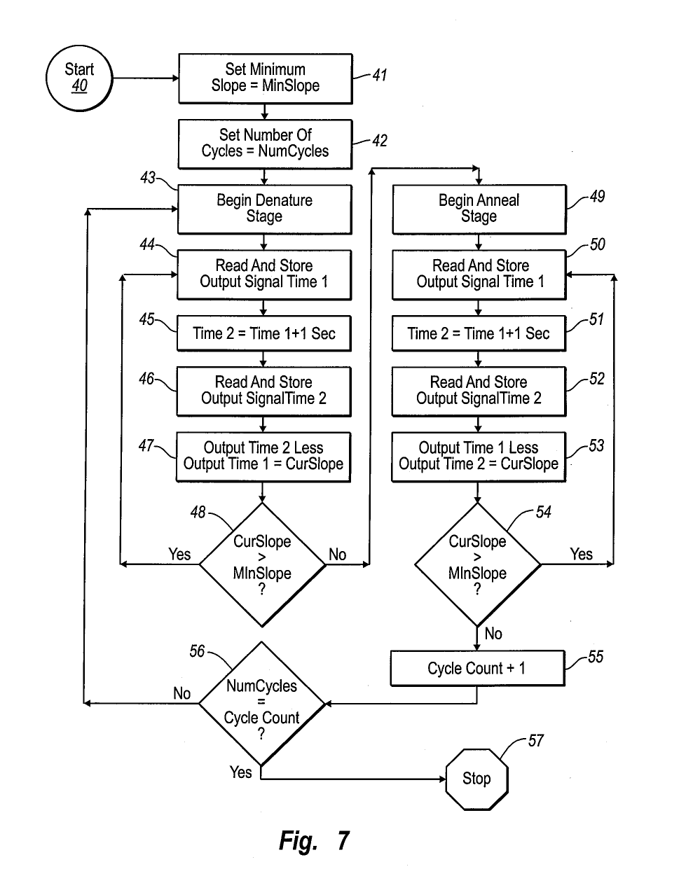

FIG. 6 is a schematic diagram showing one embodiment of the method and apparatus of the invention. A reaction vial 24 is provided to hold the DNA sample to be amplified and other substances used during the course of amplification. Reaction vial 24 is held by a reaction vial holder 26 which effects a change in temperature of the contents of reaction vial 24 from stage to stage.

A fluorescent detection system 28 advantageously stimulates and monitors fluorescent illumination and reports such illumination to a microprocessor 30, which may be part of a computer or a programmable logic controller. The microprocessor is programmed to look for events during DNA amplification, such as the reduction in the amount of change in the fluorescent illumination over time. Selection of an appropriate value for the amount of change in fluorescence illumination might depend upon factors such as how long it takes the system to react after a signal is given to move to the next stage. Since the temperature change is not effected immediately in conventional thermocyclers, it might be optimum to proceed to the next stage when the slope of illumination becomes less steep, but before it is flat, in recognition of the inherent delay from the time a signal is sent to effect a temperature change until the temperature change can actually be effected.

Once the proper test conditions are met by data monitored by microprocessor 30, a signal is sent to cycle controller 32 to direct it to go to operation of the next stage in the DNA amplification process.

Although various implementations of the present invention are contemplated, one embodiment of the invention is illustrated schematically in the logic flow diagram of FIG. 7 for a microprocessor that will analyze data from a suitable detection system, in this case a fluorescence detection system, and then pass instructions to a cycle controller when it is time to advance to the next stage or to termination cycling.

The starting point for discussing this diagram is marked with reference numeral 40, and labeled “start.” Step 41 involves the selection and entry of a number by a user, currently contemplated as being between 0 and 20 when using a fluorescent detector of the type disclosed in copending application Ser. No. 11/031,526. This number will be the maximum differential allowed when analyzing the data from the fluorescence detector before advancing to the next stage. If the user enters 0, each stage being monitored will continue until the differential in illumination from one reading to the next becomes zero. If the operator enters 20, then the stage will continue until the differential in illumination from one reading to the next becomes 20 or less. The effect of setting a lower number will be to lengthen the time in the affected stage. Conversely, a higher number will cause an earlier conclusion of the stage. The value entered by the operator is referred to in FIG. 7 as the “minimum slope” (MinSlope).

Step 42 depicts the selection and entry of the number of cycles that will be performed during DNA amplification. This number is typically determined by reference to how much amplification is desired. Most conventional thermocyclers are operated between 15 and 60 cycles. The number of cycles input in this step is referred to as the “number of cycles” (NumCycles).

Step 43 depicts the commencement of the denaturation stage. The temperature of the sample vial is raised to the appropriate temperature so that denaturation begins. Unlike conventional thermocyclers which operates on a fixed timer, the present invention monitors the progression of the denaturation stage, in this example by monitoring fluorescence. Step 44 involves taking a reading of the signal from the fluorescence detector system at (Output Signal Time1), and the result is stored in a temporary register within the computer. Step 45 involves addition of time, in this case one second, to (Output Signal Time1), and the result is stored as (Output Signal Time2). Smaller or larger slices of time may be used.

Step 46 involves taking another reading of the signal from the fluorescence detector system at (Output Signal Time2), and storing that signal in another temporary register within the computer.

Step 47 subtracts the value of the reading taken at (Output Signal Time1) from the reading taken at time (Output Signal Time2). The result is referred to in FIG. 7 as Current Slope (CurSlope). If, for example, the digital value of the fluorescent signal at (Output Signal Time1) was 3415 and at (Output Signal Time2) was 3428 then the value of (CurSlope) would be 13.

Step 48 involves a comparison of (MinSlope) to (CurSlope). If (CurSlope) is greater than (MinSlope), the logic sequence leads to a repeat of Step 44, and this loop will continue until such time as (CurSlope) becomes equal to or less than (MinSlope). Once this latter event occurs, the denaturation stage is complete, and the logic sequence moves to Step 49.

Step 49 involves directing the cycle controller to advance the process to the annealing stage. Step 50 reads the output of the fluorescent detector and records it in a temporary register within the computer at (Output Signal Time1). Step 51 increments the timer, again in this example by addition of one second to (Output Signal Time1) and this becomes (Output Signal Time2). Step 52 takes another reading from the fluorescent detector, and stores this reading at (Output Signal Time2).

Since the curve in the annealing stage is a rising curve rather than a descending curve, the math operation is inverted and the reading of (Output Signal Time2) is subtracted from the reading of (Output Signal Time1). The mathematical resultant is again (CurSlope) as depicted in Step 53 of FIG. 7. Step 54 involves a comparison between (CurSlope) and (MinSlope). When CurSlope is greater than MinSlope, the process loops back to step 50. When CurSlope is equal to or smaller than MinSlope, the process continues to step 55.

Step 55 increments the cycle counter. A comparison is then made at Step 56 of the Cycle Count to NumCycles. The process loops back to step 43 and another denaturation step until such time as the desired number of cycles have been completed, at which time the process moves to Step 57, which concludes the amplification process.

It will be appreciated that numerous modifications may be made to the process of FIG. 7 without departing from the inventive concepts of monitoring the stages of an amplification process and terminating each stage by reference to the state of reaction.

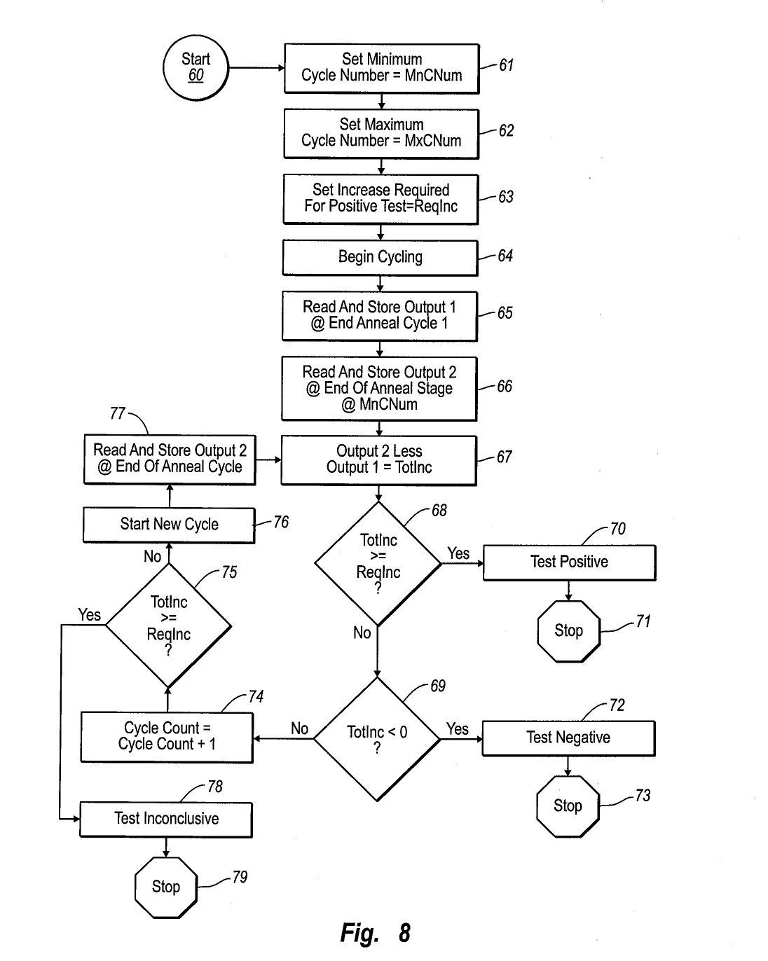

It is also possible to add even greater optimization through automation. For example, FIG. 8 illustrates a logic flow diagram for executing an automatically determining cycle count. Step 60 is the starting point for this diagram. Step 61 involves user input of the minimum number of cycles (MnCNum) that would be performed. This insures that a desired minimum number of cycles are performed. Step 61 involves user input of a maximum number of cycles (MxCNum) that would be performed if the process does not cut off the cycling earlier. Setting the minimum number of cycles and the maximum number of cycles at the same value could be used to specify a particular number of cycles.

Step 63 involves user input of a value for the minimum amount of increase in luminescence (ReqInc) which would need to be measured by reading a signal from the fluorescence detector system (compare Step 44 of FIG. 7) in order to be identified as a positive test. This number would likely be empirically derived from prior experience with the equipment and the particular fluorescence probe being used.

Cycling commences at Step 64. The output from the peak illumination is read at the end of the first cycle as Step 65, and stored as Output 1. Step 66 involves reading and storing of the output of the peak illumination at the end of the minimum number of cycles entered in MnCNum in Step 61. This value is stored as Output 2.

Step 67 involves subtracting Output 1 from Output 2. The result is Total Increase (TotInc). Step 68 compares the value of TotInc to the value of ReqInc from Step 63. In the case where TotInc is greater than or equal to ReqInc, flow will pass to Step 70. In the case where TotInc is less than ReqInc, flow will pass to Step 69.

Step 70 results in flagging Test Positive, which leads to conclusion of operation of the program at Step 71.

Step 69 makes a second comparison. When TotInc is less than zero, flow passes to Step 72. When TotInc is greater than or equal to zero, flow passes to Step 74.

Step 72 results in flagging Test Negative, which leads to conclusion of operation of the program at Step 73.

Step 74 increments the cycle count and passes flow to Step 75, which compares Cycle Count to MaxCNum, the value entered by the user in Step 62. When the cycle count is less than MaxCNum, flow continues to Step 76, which commences a new cycle. In that event, Step 77 involves reading and storing a new value for Output 2, and flow continues back to Step 67. When the test of Step 75 is satisfied, flow passes to Step 78, which flags the test as Inconclusive, and then flows to Step 79, which terminates the program.

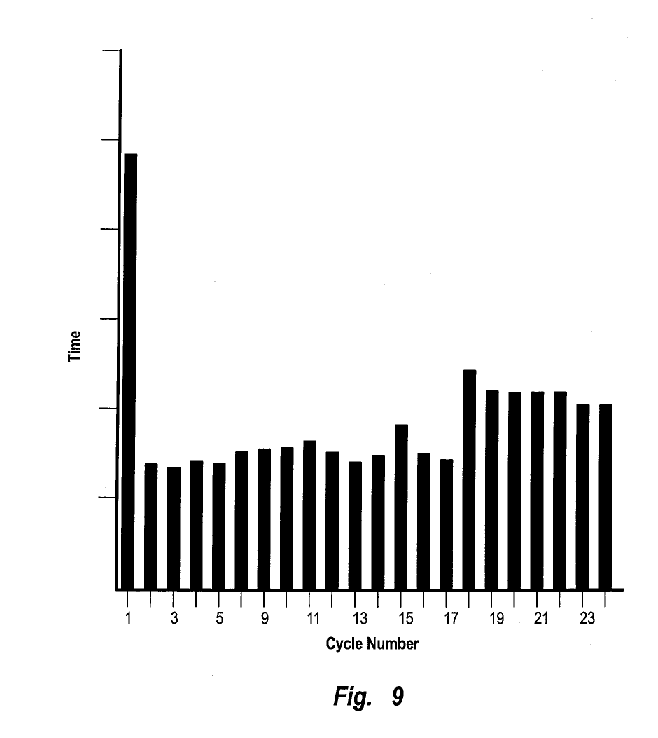

A surprising discovery was made in connection with the present invention. It has been discovered that it is not optimal for all cycles to be identical. FIG. 9 depicts the results of one DNA amplification experiment, showing the optimized time of each cycle based on monitoring of fluorescence in a two stage cycler. Cycle one was discovered to require a substantially longer cycle time than any subsequent cycle. Cycles 18 through 24 also required longer cycle time than preceding cycles. It is not known why these differences exist, but empirical evidence monitoring the completion of the denaturation and the annealing reactions clearly shows that different times are optimal from cycle to cycle. The present invention allows optimum times to be established in a closed loop system by reference to actual measurements, which takes into account in real time anything that affects completion of the desired reactions in each cycle.

In view of the foregoing, it will be appreciated that an embodiment of the invention includes a detector capable of measuring a parameter of a reaction of at least one stage of DNA amplification; a microprocessor for making comparisons in changes in that parameter over time, especially as the reaction of that stage nears completion; and a controller for terminating the current stage of DNA amplification and progressing to the next stage. Although the parameter currently preferred is fluorescence, other parameters could be measured.

Many enhancements to this basic system may be easily included. For example, one can include a user-defined endpoint measurement value, such as described above, or one could build into the apparatus the ability to automatically move from one stage to the next when the reaction has slowed, but is not complete, taking into account the inherent lag in the apparatus in changing from the current stage to the next stage of DNA amplification.

The method of the invention may include the steps of monitoring the progress of at least one stage of DNA amplification by measuring a parameter that provides data regarding the state of the reaction of that stage; determining when that reaction has reached a desired level of completion; and then progressing to the next stage of DNA amplification. This determining step can comprise observing a slow down in the reaction as evidenced by a slowing in the rate of change of the parameter being measured, or a specific difference in output from one measurement to the next, or taking into account the lag of the apparatus in changing from one stage to the next.

As noted above, for purposes of brevity the present invention has been described by reference to a two stage thermocycler and using fluorescence as a measurement tool. The invention need not be restricted to the specific methods and apparatus described; the foregoing description is merely exemplary. Real-time monitoring of the state of reaction in other DNA amplification systems, whether a two stage system, a three stage system, or a totally difference type of DNA amplification system, will allow one to control the operation of that system in an optimal manner. Such real time monitoring may of course be accomplished using fluorescence, such as described above, or may be monitored in some other fashion.

Hence, it will be appreciated that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A cycler for DNA amplification, comprising:
a detector capable of measuring a parameter of at least one stage of DNA amplification that changes as the reaction of said stage approaches completion;
a microprocessor for making comparisons in the parameter that changes as the reaction of said stage approaches completion; and
a controller for terminating said at least one stage and progressing to the next stage of DNA amplification when said microprocessor makes a comparison that corresponds to a desired state of completion of the reaction of said stage.

2. The cycler of claim 1, wherein the detector comprises a fluorescence illumination excitation source and a fluorescence receiver for detecting fluorescence during the course of DNA amplification.

3. The cycler of claim 1, wherein the microprocessor makes comparison in said parameter and sends a signal to the controller based on a user defined value based on a measurement of the parameter at one time and a measurement of said parameter at a second time.

4. The cycler of claim 1, wherein the microprocessor makes comparison in said parameter and sends a signal to the controller prior to completion of the reaction based on inherent lag in the cycler in changing from the current stage to the next stage of DNA amplification.

5. The cycler of claim 1, wherein the detector is capable of measuring a parameter of each stage of DNA amplification that changes as the reaction of said stage approaches completion.

6. The cycler of claim 5, wherein the parameter measured for at least one stage is fluorescence.
